SEQUENCE LISTING (36 sequences)

SEQ ID NO: 1 - DNA, 2226 bp, Solanum lycopersicum
  LA22CD07 promoter
cgtgcgttgcacgtttatctcttaactattttataaaatttgtacttgatagacttcctattggtagttattcatgtcaaattattttgaaaaataaaagaaattgctactataaagtgtctttggaaaatgaaaaccctttccaatctttatatagttctttttcgatgatttttgaatttctttttaatgtaattatataagtgaggtcttaaaaaaattatacatatgcatccttacttctaggatgtaacttgtaaatgtaaatatatatatagatatagaaaaggcatgtaacttgtaaatgaaaatataaatatagatatagagaagaagaaacttgatattaacatatgacaatataatggaagtatcaacgtaactccaacaatcctttgtcacaacctacatataattaatattgttattgtaataatatatgatctctttgtcatgctttccctattaaagcaatgaaatgttattagaatgactcgctgttatatatcttgcaaattaataaattacattgatagttaacgaagaagttgagggtcacattaattagtacatgacaggacattaagagaatcaatacttattggagagaagtttgatggtcacacacatcaattggtgaaactgatataattaatcaaataaattgaataacatctggaagagaattaaatgggcgtaaaaggaatgacaaatagaagttaatcttttttttatctttttttaaaacgattaattaacttttaaaggatgatatgtattatttattgttttatattatgttgatttattgtaggaataaaatggtaattcagcttttgtactttgaatttttccacttataatataataagattcttgcggcttgttgaccaataattgacaaaatatattgaaatttattgtaaataatttttttgactaaagtgcaaaacaacttcataaatatgtgtttttgaaattaaaaaaaaaaatctatattattctctaacaattacttttattattataatgtatctgtatagtccatgtagtttcattttattttatcaaagttaaaatattaatttataatttaaatatcttaacatttatcaaagactgacattttttttcttatattcagatgtttaataataaatagtcttgatcatttactgttcaaatctagagacaaaatcgtaatcactcaattgtgtattaaaatttagacatgttaatcttaatgaaaacaaatgaaacctaagctatatatgacaatacatgttgctaacatgtatcatctgacacattatttaatttttatcattgtatcttactatgtgtatatgcatcaaataaaatatacgcatgtataaatatacatatgtatctgataaaagtgtacaagtttgtatgttaaaaatctaaatatcggtgaatcgtaaaaaaaaaaagagaatgttgattgttttgtttattttcttagaagaagttgtgatatgtcagatccattataattgttataagcatgattttctcgttaattattaactttcaattatactatttaattatacacgctccttagttaatgcaaatacatataaattatatttattttgagaaattaatgagacttctttgccatgatcgaaacttgtagtaaaatttaaacgatccatcgaaatatgctttttgcaagttaagatggaattcagcaatagtgtaaactatgatttgtttgcctgttgggtgtacggaaattttaagtgagtatgattgacatacattataatgtcttttagatgttaaatttagatttcttttcaattaaagatgaaaagtcacataccaaaagtattaaactagaaatattacaatagctcaagaaataaaaatgttatttttcgttatttatttatgcaacgaatgtttaaaaatgcaaacaaaagtttcttttatataaaattgaattgaaaatatctaataaataatatctttagattaaattatcatgcttttagacgcaaaattaagatatgtaataattcgagaatcagaatgaaattatcccacctactctaaaattaaaagatacaataatttacctaaatcaaaacgacgccgtagtactgtaccttttggactttggatgttgtatctatttggaaacgaagcaactcatccttctttcataaaatcgatctagcttgatttcttccatta

SEQ ID NO: 2 - DNA, 2124 bp, Solanum lycopersicum
  LesAffx.6852.1.S1_at promoter
ttcgtggaaactatctttcacgccaacatttactgatgataagtatatgactgccatttcaaagccaaaggttgcagttatccgggaggaaggcagcaatggtgatagagaaatgtctgcagccttttctgctgctggatttgaaccatgggatgttgcaatgtcagaccttctcaatggattcatcacactggatgaatttagaggaattgtgtttgttggaggttttagttatgctgatgtacttgattctgccaaaggctggggagcgtccattcgctttaatgagcctcttctaaatcaatttcaggcattttataaccgtccggacactttcagcctcggagtttgcaatgggtgccaacttatgctctatgggtgggtgccagggccccaagtaggaggtgttttggtgccggaggggatccatcacagccaaggttcatacataatgagtctggaaggtttgaatgtcgtttcacgaatgtgacaataggaagagaaacacagccataatgttcaaaggtatggaaggtagtacattaggtgtttgggctgctcatggtgaaggaagagcttatttccctgatgatagtattttcaatcatattcttggctccaacttggcgccagtgaagtattgcaatgatgatggcacaccaacagaagtatatcctttcaatcctaacggttctcctctgggtgtagccgcaatttgttctccagatggtagacatctcgcgatgatgcctcatccagaacgctgtttcttgatgtggcagtatccatggtatcctaaaaattgggatgttgaaaagaaaggcccaagcccctggttgcgaatgtttcaaaatgccagagaatggtgctcataaggtataactctttaatttacatatttataatcaaaccctttctctcattctatctatgcatgttggaacatatatttatgctcttatcaaatgtgcagatctttcaaaaattccaaaggcgtgatatttgtgtttgacaaacgacatagaaggcactgaaggtgcagagattgagattcttgcaatttggttgcagatacgacgggacaaactaatgagtctttcgtcattcttatcgcgcctttcatcattctcaagaatatatttggttctaataccaagtattttgatgaatcaaatgcgtaactgaaactacgagcgcagttccttggacaattctgctgtctttttgacactattatggttcacccatcaaaatctgtccttaatatgcagagactcactcaattttagcaacttatttttatcctttagtctaagattaagtgcacttgtccttcacaaattgtcgattgattgaatgaactagaacttaccaatatatagctcagcgtatttttttttcgagtttacgatataactaccctcacaacttttcgagacaatcgtattacagtgtaataagtcaatttcctgccttaaccagttgctccattgcaaatactactgcatatataagtcaattttcctgtcttaatctatagctgcaaacattacttcttccaatatatcaaaagactttattttgagaaattatttctttactagtgatatatttttgtcacggtattcctaccttaattggtctaattgagctagagaaaaaaacaatctttgttttcccatcgatacattattgaagtaatttatattaacacatgttaaataccagaaaatgtgtcttatatatccaatagccacacctaccattgtacccttcctcaatcatcactttgaagttcaaaccatatatagctgaaaaaagctagcacagtttttcatgtatgtaacctacgtagagttattggaatcgcgatgtaaatattttttttataccatcagggtatctaaagatatctatataggtaggtctcctataaatgtggaatgtaaaatcttgacaatatgatgtatttttgtgttacaactaaaaataatgtgatcttgatagtgaaaaaattatttatactatccgtatacgtaacttactcttttgctagatctataaaacaatgagatcaggcctagaatgatcacaaagtcaataactgtttaatgtaactgaaaa

SEQ ID NO: 3 - DNA, 23 bp, Artificial
  primer
tcactcaccaagccctttctctc

SEQ ID NO: 4 - DNA, 23 bp, Artificial
  primer
tcctgagaagcagccttaggaac

SEQ ID NO: 5 - DNA, 20 bp, Artificial
  primer
cgatccgcgctaatcatcgt

SEQ ID NO: 6 - DNA, 20 bp, Artificial
  primer
agccgtgctctgcatctttg

SEQ ID NO: 7 - DNA, 20 bp, Artificial
  primer
tggtggtgaggctgttgagc

SEQ ID NO: 8 - DNA, 20 bp, Artificial
  primer
ccatgagtcggaacctgtgc

SEQ ID NO: 9 - DNA, 21 bp, Artificial
  primer
tggcgttttcttcatcctcca

SEQ ID NO: 10 - DNA, 23 bp, Artificial
  primer
cagctgcccttatcctgaactga

SEQ ID NO: 11 - DNA, 20 bp, Artificial
  primer
cggggtgttgatgctgaaac

SEQ ID NO: 12 - DNA, 24 bp, Artificial
  primer
gaggggcttccattcattatcaga

SEQ ID NO: 13 - DNA, 20 bp, Artificial
  primer
aaccctcgccggagcatcaa

SEQ ID NO: 14 - DNA, 24 bp, Artificial
  primer
tttaatgggatccccaacttcttg

SEQ ID NO: 15 - DNA, 23 bp, Artificial
  primer
gcaatagctggtcggctagaaca

SEQ ID NO: 16 - DNA, 20 bp, Artificial
  primer
atcgattcgctgcggcctta

SEQ ID NO: 17 - DNA, 27 bp, Artificial
  primer
atgtctttgggtggaattgtggatgcc

SEQ ID NO: 18 - DNA, 25 bp, Artificial
  primer
catctcctcgcaaagctaccagttc

SEQ ID NO: 19 - DNA, 28 bp, Artificial
  primer
atgtatgctacgaccattactggtagcc

SEQ ID NO: 20 - DNA, 25 bp, Artificial
  primer
caacccgctggattaatgagaccac

SEQ ID NO: 21 - DNA, 26 bp, Artificial
  primer
atgccgccgttgtttatcacccattc

SEQ ID NO: 22 - DNA, 28 bp, Artificial
  primer
gaaagaccaactgagcctctttcagaag

SEQ ID NO: 23 - DNA, 27 bp, Artificial
  primer
ccacttgtggaattggatgggatgttg

SEQ ID NO: 24 - DNA, 25 bp, Artificial
  primer
gatcacttggaggagctgtatagcc

SEQ ID NO: 25 - DNA, 26 bp, Artificial
  primer
atgccgccgttgtttatcacccattc

SEQ ID NO: 26 - DNA, 28 bp, Artificial
  primer
gaaagaccaactgagcctctttcagaag

SEQ ID NO: 27 - DNA, 20 bp, Artificial
  primer
aactccatgcggggagtcat

SEQ ID NO: 28 - DNA, 22 bp, Artificial
  primer
ggcctctgttatggaggtaact

SEQ ID NO: 29 - DNA, 22 bp, Artificial
  primer
aactgggatgatatggagaaga

SEQ ID NO: 30 - DNA, 21 bp, Artificial
  primer
tctcaacataatctgggtcat

SEQ ID NO: 31 - DNA, 30 bp, Artificial
  primer
gggaagctttcgtggaaactatctttcacg

SEQ ID NO: 32 - DNA, 36 bp, Artificial
  primer
ggggtctagattttcagttacattaaacagttattg

SEQ ID NO: 33 - DNA, 23 bp, Artificial
  primer
atgcaagcttcgtgcgttgcacg

SEQ ID NO: 34 - DNA, 27 bp, Artificial
  primer
atgcggatcctaatggaagaaatcaag

SEQ ID NO: 35 - DNA, 26 bp, Artificial
  primer
gatcagttcgcccatgcagatattcg

SEQ ID NO: 36 - DNA, 22 bp, Artificial
  primer
cttgcaaagtcccgctagtgcc

(19) United States
(12) Patent Application Publication
(10) Pub. No.: US 2014/0020134 A1
(43) Pub. Date: Jan. 16, 2014

(54) FRUIT-SPECIFIC PROMOTER

(71) Applicants: Nobuyo Itou, Yokohama-shi (JP); Hirofumi Kuroda, Yokohama-shi (JP); Ken-ichi Takane, Yokohama-shi (JP); Kou Aoki, Kisarazu-shi (JP); Daisuke Shibata, Kisarazu-shi (JP); Hiroshi Ezura, Tsukuba-shi (JP); Kyoko Tanase, Tsukuba-shi (JP)

(72) Inventors: Nobuyo Itou, Yokohama-shi (JP); Hirofumi Kuroda, Yokohama-shi (JP); Ken-ichi Takane, Yokohama-shi (JP); Kou Aoki, Kisarazu-shi (JP); Daisuke Shibata, Kisarazu-shi (JP); Hiroshi Ezura, Tsukuba-shi (JP); Kyoko Tanase, Tsukuba-shi (JP)

(73) Assignees: INPLANTA INNOVATIONS INC., Yokohama-shi, Kanagawa (JP); UNIVERSITY OF TSUKUBA, Tsukuba-shi, Ibaraki (JP); KAZUSA DNA RESEARCH INSTITUTE, Kisarazu-shi, Chiba (JP)

(21) Appl. No.: 14/007,813

(22) PCT Filed: Mar. 30, 2011
(86) PCT No.: PCT/JP2011/058031

§ 371 Date: Sep. 26, 2013

(51) Int. Cl. C12N 15/113

(52) U.S. Cl. CPC C12N 15/113
USPC 800/287; 536/24.1; 435/320.1; 435/419; 800/298

(57) ABSTRACT

The present invention relates to a fruit-specific promoter, which is suitable for the expression in a broader range of developmental stages of a fruit. Provided is a fruit-specific promoter DNA, which consists of a nucleotide sequence having 85% or more identity with the nucleotide sequence as shown in SEQ ID NO: 1 or 2 and has promoter activity in mature-green fruits.

### TECHNICAL FIELD

The present invention relates to a fruit-specific promoter.

### BACKGROUND ART

Production of transformed plants often use the constitutive promoter CaMV-derived 35S promoter in order to induce expression of target genes in plants. Also, promoters inducing gene expression specific to particular developmental stages or particular tissues of plants have been isolated and utilized for production of transformed plants for which limited gene expression is desirable. For example, the promoter of the E8 gene isolated from tomato is known to induce expression of a target gene in fruits in a manner specific to the after-ripening stage (Deikman et al., EMBO J., 7: 3315-3320, 1988). However, conventional fruit-specific promoters do not function in early developmental stages of fruits.

International Publication WO 96/14421 discloses successful induction of gene expression throughout fruit developmental stages of transformed tomato using a promoter isolated, from potato. This document, however, further discloses that the potato-derived promoter also induced gene expression in leaves or stems of tomato, and in tubers of potato.

That is, it has been found that the potato-derived promoter is not capable of inducing fruit-specific gene expression.

### REFERENCES

### Patent Documents

Patent Document 1: International Publication WO 1996/14421

### Non-Patent Documents

Non-Patent Document 1: Deikman et al., 1988, the EMBO Journal 7: 3315-3320

### SUMMARY OF THE INVENTION

### Problem to be Solved by the Invention

It is an object of the present invention to provide a fruit-specific promoter that is capable of inducing gene expression throughout a broader range of developmental stages of fruits.

### Problem to be Solved by the Invention

The present inventors have conducted concentrated studies in order to solve the above object, and then succeeded in isolating a fruit-specific promoter that functions in mature-green fruits in addition to red-ripe fruits of tomato, and completed the present invention.

Specifically, the present invention includes the followings.

[1] A fruit-specific promoter DNA, which consists of a nucleotide sequence having 80% or more identity with the nucleotide sequence as shown in SEQ ID NO: 1 or 2 and having promoter activity in mature-green fruits.

According to a preferred embodiment, further, the fruit-specific promoter DNA may consist of a nucleotide sequence having 90% or more identity with the nucleotide sequence as shown in SEQ ID NO: 1 or 2.

[2] An expression vector comprising the fruit-specific promoter DNA according to [1] above.

The expression vector may further comprise a gene ligated downstream of the fruit-specific promoter DNA according to [1] above.

[3] A DNA construct comprising the fruit-specific promoter DNA according to [1] above and a gene ligated downstream thereof.

[4] A transformed cell comprising the expression vector according to [2] above or the DNA construct according to [3] above.

[5] A transformed plant into which the expression vector according to [2] above or the DNA construct according to [3] above is introduced.

[6] A method for producing a transformed plant comprising introducing the expression vector according to [2] above or the DNA construct according to [3] above into a plant, growing the transformed plant to develop a fruit, and confirming expression of the gene in the fruit.

[7] A method for producing a recombinant gene product comprising growing the transformed plant according to [5] above to develop a fruit and obtaining the expressed gene product from the fruit.

### Effects of the Invention

With the use of the promoter of the present invention, the expression of a foreign gene can be induced in a broader range of developmental stages of a fruit.

### BRIEF DESCRIPTION OF THE DRAWINGS

FIG. 1 is a photograph showing the results of RT-PCR analysis of expression in mature-green fruits of candidate high expression genes in mature-green fruits. Genes showing high expression are marked with asterisks.

FIG. 2 is a photograph showing the results of RT-PCR analysis of expression in a variety of tissues of candidate fruit-specific genes. Genes showing high expression in mature-green fruits are marked with asterisks.

FIG. 3 is a photograph showing the results of analysis of transformed plants via histochemical tissue staining. FIG. 3A shows the results of overnight (16 hours) staining using 100 mM phosphate buffer (pH 8.0) as a reaction solution. FIG. 3B shows the results of staining for 6 hours using a reaction solution supplemented with 20% methanol.

### EMBODIMENTS FOR CARRYING OUT THE INVENTION

Hereafter, the present invention is described in detail.

The present invention relates, to a promoter that is capable of inducing fruit-specific gene expression starting from early fruit developmental stages before the full-maturity stage.

More specifically, the promoter according to the present invention is fruit-specific promoter DNA consisting of a nucleotide sequence having 80% or more identity with the nucleotide sequence as shown in SEQ ID NO: 1 or 2 and having promoter activity in mature-green fruits.

In the context of the present invention the phrase “a nucleotide sequence having 80% or more identity with the nucleotide sequence as shown in SEQ ID NO: 1 or 2” means a nucleotide sequence having 80% or more, preferably 90% or more, more preferably 95% or more, further preferably 97% or more, and particularly preferably 99% or more (e.g., 99.5% or more) identity with the full-length of either of the nucleotide sequence as shown in SEQ ID NO: 1 or 2 when aligned with such sequence. The alignment of nucleotide sequence can be performed using, for example, the multiple sequence alignment program Clustal W (Thompson, J. D. et al., 1994, Nucleic Acids Res., 22, pp. 4673-4680; available from the website of the DNA Data Bank of Japan (DDBJ) or that of the European Bioinformatics Institute (EBI)) at default settings, or performed manually.

Alternatively, the promoter according to the present invention may be a fruit-specific promoter DNA consisting of a nucleotide sequence derived from the nucleotide sequence as shown in SEQ ID NO: 1 or 2 by deletion, substitution, and/or addition of, for example, 1 to 50, preferably 1 to 10, and more preferably 1 to 5 nucleotides and having promoter activity in mature-green fruits.

In the context of the present invention, the term “mature-green fruit(s)” refers to a relatively immature fruit at the mature-green stage. In agricultural fields, the stage at which fruit growth is completed is referred to as the “mature-green stage.” Fruits at the mature-green stage have not developed full color and they are generally green, pale green, or pale yellow. The stage during which a fruit ripens after the mature-green stage is referred to as the after-ripening stage. During the after-ripening stage, fruits show changes; for example, develop sweetness and flavor, and undergo pulp softening. In general, some fruits, such as tomatoes, apples, bananas, and pears, are harvested during the mature-green stage, the harvested fruits subsequently ripen and develop full color, and fully-ripened ready-to-eat fruits are then shipped to market. Fully-ripened fruits of tomato subjected to ripening (on the tree or after harvest), following the mature-green stage, are referred to as “red-ripe fruits.” Fully-ripened fruits of other fruits that have developed full color and matured are also referred to as “red-ripe fruits” or “ripened fruits.” Herein, “red-ripe fruits” or “ripened fruit” may be red in color, or may be actually orange, purple, yellow, white, or the like in color.

The promoter according to the present invention has promoter activity in fruits at the mature-green stage (i.e., mature-green fruits). Particularly preferably, the promoter according to the present invention has promoter activity in fruits at the mature-green stage, in addition to after-ripening stages including the full-maturity stage. In the present invention, the term “promoter activity” refers to the ability to induce expression of a gene (typically a constitutive gene) under the control of a promoter (typically, a gene located immediately downstream of the promoter). The phrase “ . . . induce expression of a gene” means that the promoter causes the initiation of production of a transcript (mRNA) from the gene of interest.

The promoter according to the present invention has strong promoter activity in mature-green fruits of a wide variety of fruit plants. A typical example is a promoter having strong promoter activity in mature-green tomato fruits. Tomato includes, but not limited to, cultivars Micro-Tom and Moneymaker, for example.

A particularly preferred promoter according to the present invention is DNA consisting of the nucleotide sequence as shown in SEQ ID NO: 1 or 2. DNA of the nucleotide sequence as shown in SEQ ID NO: 1 or 2 is a tomato-derived promoter and is capable of inducing particularly high gene expression in mature-green fruits, as well as inducing gene expression in fruits at the after-ripening stage, e.g., fully-ripened fruits (referred to as red-ripe fruits, for tomato).

The promoter according to the present invention is capable of inducing fruit-specific gene expression in fruit plants. In the context of the present invention, the term “fruit-specific” used for promoter activity means that the promoter induces expression at markedly higher levels in fruits compared with other plant tissues. The fruit-specific promoter according to the present invention provides a gene expression level that is typically 5 or more times higher, preferably 10 or more times higher, and further preferably 100 or more times higher in fruits (e.g., fruit pulp) than in other plant tissues. Preferably, the fruit-specific promoter according to the present invention induces strong gene expression in fruits, whereas it induces no or substantially no gene expression be induced in other plant tissues that are not associated with fruit development (e.g., leaves, stems, or roots). In addition to the strong induction of gene expression in fruits, the promoter according to the present invention may induce weak gene expression in other plant tissues associated with fruit development, such as flowers. Such promoter is also regarded as having fruit-specific promoter activity.

Fruit plants, which can be used for the induction of gene expression using the promoter according to the present invention, may be any fruit plants fully ripening after passing through the mature-green stage, and include for example, but not limited to, tomato, apple, pear, banana, strawberry, melon, citrus fruits (e.g., grapefruit, orange, or Citrus unshiu), kiwi fruit, peach, blueberry, and grape.

The promoter according to the present invention can be isolated via PCR amplification using the genome of any fruit plant (such as tomato) as a template as described in the Examples below. Alternatively, the promoter can be obtained by hybridizing restriction enzyme-cleaved fragments of such genome to the promoter DNA or a part thereof as a probe. The obtained promoter DNA is preferably extracted and purified in accordance with conventional techniques. In addition, the promoter according to the present invention can be constructed by joining chemically synthesized DNA fragments designed on the nucleotide sequence information of the promoter (SEQ ID NO: 1 or 2) together.

Further, the promoter according to the present invention may be produced by modification of the nucleotide sequence of the obtained promoter DNA through mutagenesis techniques, such as site-directed mutagenesis. Mutagenesis can be performed by a conventional technique such as the Kunkel method or the Gapped duplex method, or by another technique based on these techniques. A person skilled in the art would readily perform such mutagenesis techniques using, for example, a commercially available site-directed mutagenesis kit (e.g., Mutan®-K, Mutan®-Super Express Km, or PrimeSTAR®Mutagenesis Basal Kit (Takara Bio Inc.)).

The obtained promoter DNA may be preferably subjected to nucleotide sequence determination in order to confirm whether a promoter of interest has been obtained. Nucleotide sequence determination can be carried out by known techniques, such as the Maxam-Gilbert sequencing or dideoxynucleotide chain termination, and it may generally be carried out with the use of an automated nucleotide sequencer (e.g., ABI DNA Sequencer).

According to the present invention, the promoter may be integrated into any vector to produce a fruit-specific expression vector. Thus, the present invention also provides a vector, and, in particular, an expression vector, comprising the promoter according to the present invention.

The vector into which the promoter according to the present invention is to be integrated may be any vector without particular limitation, provided that such vector is replicable in a host cell, and includes, for example, plasmid DNA and phage DNA. Examples of the plasmid DNA include E. coli-derived plasmids (e.g., pET22b(+), pBR322, pBR325, pUC118, pUC119, pUC18, pUC19, pBluescript, and pET100/D-TOPO), Bacillus subtilis-derived plasmids (e.g., pUB110 and pTP5), and yeast-derived plasmids (e.g., YEp13, YCp50, and pPICZαA). Examples of phage DNAs include λ phages (e.g., Charon 4A, Charon 21A, EMBL3, EMBL4, λgt10, λgt11, λZAP, and λZAPII). In addition, animal virus vectors, such as retrovirus or vaccinia virus vectors, and insect virus vectors, such as baculovirus vectors, can also be used. The promoter according to the present invention may be integrated into a vector by, for example, cleaving the terminus of a DNA fragment comprising the promoter with an adequate restriction enzyme, and inserting the cleaved fragment into and ligated to the restriction enzyme site or multicloning site of the vector DNA.

When the Agrobacterium method is employed, an expression vector such as a binary vector, suitable for the Agrobacterium method or a modified vector thereof is preferably used as a plant expression vector into which the promoter according to the present invention is to be integrated. Examples of such plant expression vectors include pBI121, pBIN19, pSMAB704, pCAMBIA, and pGreen. For example, the expression vector according to the present invention may be produced by substituting the promoter in such a vector with the promoter according to the present invention.

The promoter according to the present invention may be used in combination with a variety of terminator (preferably, but not limited to, plant terminator) in the expression vector. The expression vector according to the present invention may comprise a gene insertion site downstream of the promoter according to the present invention.

The expression vector can be preferably used as a tool for expressing a gene product, for example, a protein encoded by a gene of interest in a manner specific to plant fruits.

The expression vector according to the present invention may further comprise a gene for expression in a gene insertion site (e.g., a restriction enzyme cleavage site) under the control of the promoter according to the present invention. Typically, the expression vector according to the present invention may comprise a gene for expression that is ligated to downstream of the promoter DNA according to the present invention.

The gene for expression is not particularly limited, and the gene may be a gene encoding a protein or RNA, or a gene encoding a fusion protein of two or more proteins. One gene or two or more genes may be used as the gene for expression. Since the expression vector according to the present invention induces fruit-specific expression, the gene for expression is preferably a nucleic acid encoding a gene product (a protein or RNA) that is desired to be expressed, accumulated, and/or produced in fruits. Examples of the gene for expression include genes encoding any peptides or proteins, such as miraculin, various types of vaccine antigens for humans or domestic animals, cytokines (e.g., interferon and interleukin), enzymes (e.g., DNA polymerase, RNA polymerase, amyloglucosidase, amylase, invertase, isoamylase, protease, papain, pepsin, rennin, cellulase, pectinase, lipase, lactase, glucose oxidase, lysozyme, glucose isomerase, chymotrypsin, trypsin, cytochrome, seaprose, serratio peptidase, hyaluronidase, bromelin, urokinase, hemocoagulase, thermolysin, and urease), hormone proteins (e.g., insulin, glucagon, secretin, gastrin, cholecystokinin, oxitocin, vasopressin, growth hormone, thyroid stimulating hormone, prolactin, luteinizing hormone, follicle stimulating hormone, adrenocorticotropic hormone, thyroid stimulating hormone releasing hormone, luteinizing hormone releasing hormone, corticotrophin releasing hormone, growth hormone releasing hormone, and somatostatin), opioid peptides (e.g., endorphin, enkephalin, and dynorphin), blood coagulation factors (e.g., fibrinogen and prothrombin), and protease inhibitors (e.g., SSI). The gene for expression in the present invention may be derived from any organisms (e.g., plants, animals, fungi, and bacteria) or viruses, or may be artificially prepared genes. In the context of the present invention, “the gene (for expression)” is not limited to a naturally occurring gene, and the gene may be any nucleotide sequence encoding a peptide, polypeptide, protein, or RNA.

The expression vector according to the present invention may further comprise a useful sequence, such as a selection marker gene or a reporter gene indicating that a vector is retained in a cell, a polylinker for easy insertion of a gene in correct orientation into a vector, a poly(A) addition sequence, a secretory signal sequence, or a histidine tag sequence for purification, as appropriate. Examples of selection marker genes include, for example, dihydrofolate reductase gene, hygromycin resistance gene, ampicillin resistance gene, kanamycin resistance gene, neomycin resistance gene, and chloramphenicol resistance (CAT) gene.

The present invention also provides a DNA construct comprising the gene for expression under the control of the promoter according to the present invention. The DNA construct typically comprises the promoter DNA according to the present invention and a gene ligated to downstream of the promoter DNA. The term “DNA construct” used in the present invention refers to a DNA fragment obtainable by joining two or more functional DNA units (e.g., gene, promoter, and terminator) and incapable of autonomous replication, e.g., a gene expression cassette. The DNA construct according to the present invention may further comprise other functional units, such as a terminator, in addition to the promoter DNA and the gene ligated to downstream of the promoter DNA.

According to the present invention, a transformant (e.g., a transformed cell or transformed plant) can be produced by introducing the expression vector or DNA construct into a host. Specifically, for example, the expression vector or DNA construct according to the present invention can be introduced into a host cell to produce a transformed cell comprising the expression vector or DNA construct. By introducing the expression vector or DNA construct according to the present invention into a host cell (preferably a plant cell), a DNA fragment comprising the promoter and the gene for expression under the control thereof may be integrated into the genome of the host cell. Alternatively, in a transformant comprising the expression vector according to the present invention introduced into a host cell, the expression vector may be extrachromosomally maintained e.g., in the cytoplasm and the gene for expression may be extrachromosomally, transiently expressed. Any host cells, such as bacterial cells, e.g., Escherichia coli or Bacillus subtilis, yeast cells, insect cells, animal cells (e.g., mammalian animal cells), or plant cells, may be used. In the contest of the present invention, plant cells, in particular angiosperm cells, preferably cells of fruit plants, and more preferably tomato cells, can be used as host cells. Host cells may be derived from any tissues, and may be from leaves or fruits. The transformed plant cells are, preferably, for example, cells of the plant into which a gene is to be introduced, as described below.

In the present invention, a transformed plant is preferably produced by introducing the expression vector or DNA construct according to the present invention into a plant.

A plant into which the expression vector or DNA construct according to the present invention is to be introduced may be, but not particularly limited to, preferably fruit plants, and particularly preferably fruit plants that develop fully-ripened fruits after passing through the mature-green stage. Specific examples of such plants include, but not limited to, tomato (Solanum lycopersicum), apple (Malus), banana (Musa), pear (Pyrus communis), strawberry (Fragaria L.), melon (Cucumis melo), kiwi fruit (Actinidia deliciosa), peach (Amygdalus persica), blueberry (Vaccinium myrtillus), grape (Vitis), and citrus fruits such as grapefruit (Citrus×paradisi), orange (Citrus sinensis), and Citrus unshiu (Citrus unshiu). Among them, tomato is particularly preferably used.

The expression vector or DNA construct according to the present invention can be introduced into a plant via a common plant transformation technique, such as the Agrobacterium method, the particle gun method, electroporation, the polyethylene glycol (PEG) method, microinjection, or protoplast fusion. As for details of the plant transformation techniques, a person skilled in the art can refer to the descriptions in commonly-used laboratory manuals, e.g., “Shinpan Model shokubutsu no jikken protocol, Idengakutekishuho kara genome kaiseki made” (New edition, “Experimental Protocol for Model Plants; From Genetic Technique to Genome Analysis”) (Supervised by Shimamoto K and Okada K, Shujunsha Co., Ltd., 2001), or literatures such as Hiei Y. et al., “Efficient transformation of rice (Oryza sativa L.) mediated by Agrobacterium and sequence analysis of the boundaries of the T-DNA,” Plant J., 1994, 6, 271-282 or Hayashimoto A. et al., “A polyethylene glycol-mediated protoplast transformation system for production of fertile transgenic rice plants,” Plant Physiol., 1990, 93, 857-863.

When the Agrobacterium method is employed, the expression vector according to the present invention prepared with the use of a vector suitable for the Agrobacterium method may be introduced into an adequate Agrobacterium strain, such as Agrobacterium tumefaciens, via e.g., electroporation, and a plant cell, callus, or cotyledon slice may be inoculated and infected with the transformed Agrobacterium. Examples of preferred Agrobacterium strains include, but are not limited to, GV3101, C58, C58C1R1f®, EHA101, EHA105, AGL1, and LBA4404.

The particle gun method or electroporation may be carried out using either the expression vector or the DNA construct according to the present invention. A plant slice of leaf or the like may be used as a host sample into which the expression vector is to be introduced, or a protoplast may be prepared and used for it (Christou P, et al., Bio/technology, 1991, 9: 957-962). According to the particle gun method, for example, metal particles coated with the expression vector or DNA construct according to the present invention can be delivered into the samples using a gene delivery system (e.g., PDS-1000, BIO-RAD) in accordance with the manufacturer's instructions, thereby introducing the expression vector or DNA construct into the plant cell, and obtaining transformed plant cells. In general, such procedure is carried out at a pressure of approximately 450 psi to 2000 psi at a distance of approximately 4 to 12 cm.

Subsequently, plant cells, cotyledon slices or the like into which the expression vector or DNA construct according to the present invention has been introduced are cultured in a selection medium in accordance with a conventional technique for plant tissue culture, the surviving calli are cultured on a regeneration medium containing plant hormones at adequate concentrations (e.g., auxin, cytokinin, gibberellin, abscisic acid, ethylene, or brassinoride), and thereby whole plant transformed with the expression vector or DNA construct according to the present invention can be regenerated. Thus, transformed plants can be obtained.

Whether or not the expression vector or DNA construct according to the present invention has been surely introduced into the plant is confirmed by, for example, PCR, Southern hybridization, Northern hybridization, or Western blotting. For example, genomic DNA extracted from leaves of the transformed plant may be subjected to PCR amplification using primers specific to the promoter or the integrated gene for expression within the expression vector or DNA construct according to the present invention. The resulting amplified product may be subjected to agarose gel electrophoresis, polyacrylamide gel electrophoresis, capillary electrophoresis, or the like and then stained with ethidium bromide, a SYBR Green solution, or the like. The amplified product may be detected as a clear band, thereby confirming introduction of the expression vector or DNA construct according to the present invention. Alternatively, the PCR-amplified product may be bound to a solid phase, such as a microplate, and the amplified product may be detected based on the fluorescence or enzyme reaction or the like. Concerning the produced transformed plant, preferably, activity of the gene product from the gene for expression in the expression vector or DNA construct according to the present invention is confirmed.

In the preferred transformed plant obtainable as described above, into which the expression vector or DNA construct according to the present invention has been introduced, expression of the gene for expression is strongly induced, its gene product (RNA such as mRNA or rRNA, or a protein) is produced with high efficiency, and preferably accumulated, in fruits. In the transformed plant according to the present invention, expression of the gene for expression is strongly expressed, in particular, in fruits at the mature-green stage (i.e., mature-green fruits). In the transformed plant according to the present invention, in particular, expression of the gene for expression is strongly induced throughout the fruit developmental stages from the mature-green stage to the full-maturity stage. In one preferred embodiment, in the transformed plant according to the present invention, expression of the gene for expression is weakly induced also in the flowers, which correspond to the earliest fruit developmental stage. In contrast, no or substantially no expression of the gene for expression is induced in plant tissues such as leaves, stems, or roots, in the transformed plant according to the present invention. For example, in tomato into which the gene for expression has been introduced under the control of the promoter according to the present invention, very strong expression of the gene is induced in mature-green fruits, in addition to expression of the gene induced in red-ripe fruits (i.e., fully ripened fruit). While weak expression is induced also in the flowers of the tomato, preferably, no expression of the gene is induced in plant tissues such as leaves, stems, or roots.

Thus, the present invention also provides a method for expressing a gene for expression in the above-mentioned expression vector or DNA construct in a plant in a fruit-specific manner comprising introducing the expression vector or DNA construct into the plant and growing the resulting transformed plant.

The present invention also provides a method for producing a transformed plant comprising introducing the above-mentioned expression vector or DNA construct into a plant, growing the transformed plant to develop a fruit, and confirming the expression of the gene in the fruit.

Gene expression in fruits can be confirmed using a conventional technique. For example, the gene product of the gene for expression (e.g., RNA or a protein) contained in the fruits may be detected or measured. For example, total RNA extracted from fruits may be subjected to RT-PCR analysis and amplification of mRNA from the gene for expression can be detected. Alternatively, when a reporter gene encoding a fluorescent protein or chromogenic enzyme protein is used as one of the genes for expression, production of a reporter protein in the fruits is examined via fluorescent detection or staining, and induction of the gene expression in fruits can be confirmed based thereon. Further, for the same purpose, a protein extract from the fruits may be subjected to Western blot analysis using an antibody generated against the gene product of the gene for expression as an antigen.

The present invention also provides a method for producing a recombinant gene product comprising growing the transformed plant to develop a fruit and obtaining the expressed gene product of the gene for expression from the fruit.

When the resulting gene product is a protein, the protein can be obtained from the fruit by using biochemical techniques commonly employed for protein isolation and purification, such as ammonium sulfate precipitation, gel chromatography, ion-exchange chromatography, or affinity chromatography alone or a combination thereof, as appropriate. When a gene product is RNA such as mRNA, such RNA can be isolated and purified from the fruit by a general RNA extraction technique. However, a gene product solution, for example, a solution obtained by subjecting a sample solution corrected or concentrated via e.g., centrifugation or an ultrafiltration membrane to ammonium sulfate fraction and then dialysis may be directly used.

Molecular biological/biochemical experimental procedures, such as those for preparation of DNA, PCR, ligation into a vector, cell transformation, DNA nucleotide sequencing, primer synthesis, mutagenesis, or protein extraction, that may be used in the present invention, can basically be carried out in accordance with commonly-used laboratory manuals. An example of the laboratory manuals is Sambrook et al., Molecular Cloning, A laboratory manual, 2001, Eds., Sambrook, J. & Russell, D. W., Cold Spring Harbor Laboratory Press.

### EXAMPLES

Hereafter, the present invention is described in greater detail with reference to the following examples, but the technical scope of the present invention is not limited to these examples.

### Example 1

### Selection of Candidate Genes from Microarray Data

On the basis of the microarray data obtained by hybridizing probes derived from mRNAs extracted from mature-green fruits of the tomato (Solanum lycopersicum) cultivar Micro-Tom to the Micro-Tom EST microarray, genes which showed high expression were selected as candidate high expression genes in mature-green fruits. The selected candidate genes were designated as ID Nos. LA15CA04, LA22CD07, LC09AH08, LC04DC11, LA12AA05, LA14AD08, and FB14 DB02.

Further, on the basis of the microarray data for each Micro-Tom tissue type stored in the microarray databases of the Kazusa DNA Research Institute (Chiba, Japan) and Cornell University (NY, United States), five genes exhibiting fruit-specific expression were selected as candidate fruit-specific genes. The selected candidate genes were designated as ID Nos. Les.331.1S1_at, Les.3122.2.A1_a_at, and LesAffx.6852.1.S1_at (Kazusa DNA Research Institute) and TC115787 and TC116003 (Cornell University).

### Example 2

### Extraction of Total RNA and Expression Analysis Via RT-PCR

The candidate genes selected in Example 1 were subjected to expression analysis via RT-PCR. At the outset, total RNA for RT-PCR was extracted from a biological sample of a 3-month-old tomato (Solanum lycopersicum) cultivar Micro-Tom, by the procedure described below. First, leaves, flowers, stems, roots, mature-green fruits, and red-ripe fruits were sampled from Micro-Tom in amounts of approximately 1 g each and the samples were independently frozen with liquid nitrogen. The frozen tissue samples were independently ground in a mortar to prepare powders. The powdered samples were each independently transferred to 50-ml plastic tubes, about 10 ml of TRIzol® (Invitrogen, USA) was added thereto, and then mixed with a vortex mixer for 2 to 3 minutes. The resultants were incubated at room temperature for 10 minutes, and 1 ml of chloroform was added to each tube, followed by vortex mixing for 2 or 3 minutes. Following the incubation at room temperature for 1 minute, the solution was subjected to centrifugation at 10,000 rpm for 10 minutes at 4° C. The upper layer (an aqueous phase) from each tube was transferred into a different fresh 50-ml plastic tube, an equal amount of a phenol/chloroform/isoamyl alcohol solution was added thereto, and then vortex mixed for 2 to 3 minutes. The tubes were each incubated at room temperature for 2 to 3 minutes and then subjected to centrifugation at 10,000 rpm for 10 minutes at 4° C. The upper layer (an aqueous phase) from each tube was transferred into a fresh 50-ml plastic tube, an equal amount of a phenol/chloroform solution was added thereto, and then vortex mixed for 2 to 3 minutes. The tubes were incubated at room temperature for 2 to 3 minutes and then subjected to centrifugation at 10,000 rpm for 10 minutes. The upper layer (an aqueous phase) from each tube was transferred into a fresh 50-ml plastic tube, an equal amount of isopropanol was added thereto, and then vortex mixed for 2 to 3 minutes. The tubes were incubated at room temperature for 5 minutes and then subjected to centrifugation at 10,000 rpm for 10 minutes at 4° C. The supernatant was removed, 70% ethanol cooled to −20° C. was added to the pellet, and the pellet was washed. After centrifugation of the resultant at 10,000 rpm for 10 minutes at 4° C., the supernatant was removed. After completely removing ethanol from the pellet by incubation at room temperature for 5 to 10 minutes, the pellet was dissolved in RNase- and DNase-free sterile water to prepare an RNA solution. The amount of RNA in the RNA solution was determined by measuring the OD 260 using the Satire microplate reader (Tecan, Switzerland). Subsequently, reverse transcription reaction was carried out using 1 μg of total RNA treated with DNase as a template and a poly-T primer and SuperScript® II (Invitrogen, USA) to prepare first-strand cDNA.

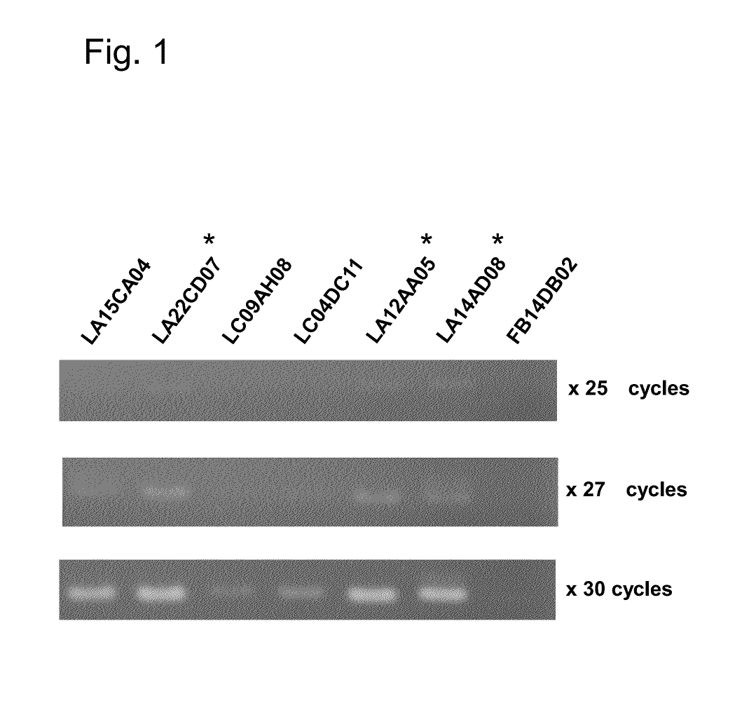

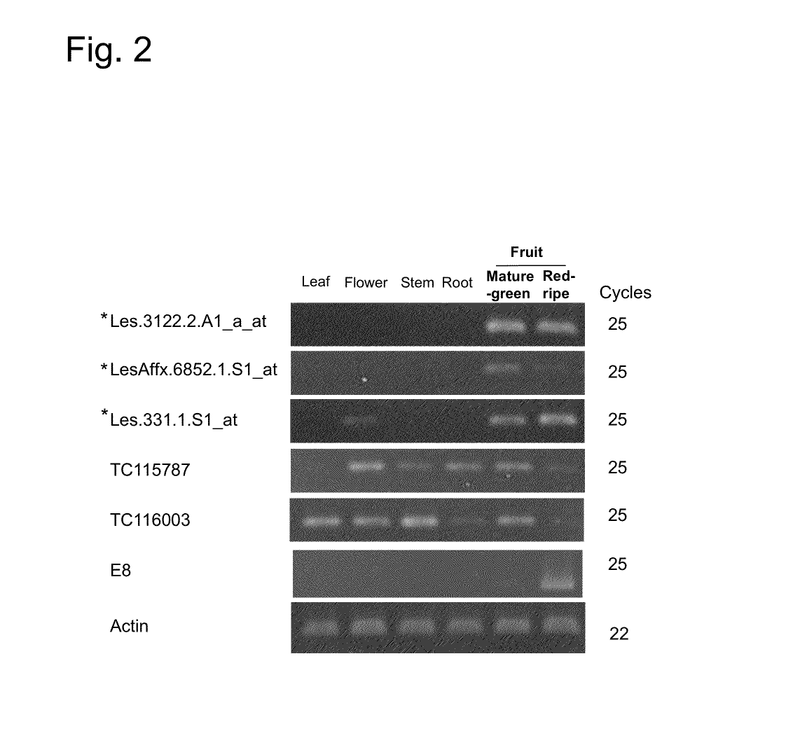

Subsequently, PCR amplification of each gene was carried out using, as a template, the first-strand cDNAs prepared from mRNAs from the leaves, flowers, stems, roots, mature-green fruits, and red-ripe fruits in the manner described above, in order to analyze the expression of candidate high expression genes in mature-green fruits. Table 1 shows primer sets used for amplification of each gene and the expected amplification sizes. As a control, the E8 gene and the actin gene were amplified by PCR. The PCR reaction was carried out in total volume of 50 μl. The PCR reaction solution was composed of 20 pM of each primer of the primer set, 1 μl of cDNA, 5 μl of 10×PCR buffer, 4 μl of dNTPs, 0.2 μl of polymerase, and 39.3 μl of sterile water. 10×PCR buffer, dNTPs, and polymerase used were from the TAKARA Taq Hot Start Version. PCR reaction was carried out under thermal cycling conditions of thermal denaturation at 95° C. for 5 minutes, followed by 20 to 35 cycles of denaturation at 94° C. for 0.5 minutes, annealing at 55° C. for 0.5 minutes, and extension at 72° C. for 1 minute. The resulting PCR product (5 μl) was electrophoresed on agarose gel, and the amplified product having the expected amplification length was detected. FIG. 1 and FIG. 2 show the results of electrophoresis. Table 1 shows the expression conditions of genes observed.

TABLE 1

| | | Result of | | | |
|---|---|---|---|---|---|
| | | RT-PCR | Primer set | | expected |
| | | expression | Forward primer for | Reverse primer for | amplification |
| Category | ID | analysis | RT-PCR | RT-PCR | length (bp) |
| High | LA15CA04 | Low | 5′-TCACTCACCAAGCC | 5′-TCCTGAGAAGCAGC | 208 |
| expression | | expression | CTTTCTCTC-3′ | CTTAGGAAC-3′ | |
| gene in | | | (SEQ ID NO: 3) | (SEQ ID NO: 4) | |
| mature- | LA22CD07 | High | 5′-CGATCCGCGCTAAT | 5′-AGCCGTGCTCTGCA | 205 |
| green | | expression | CATCGT-3′ | TCTTTG-3′ | |
| fruits | | | (SEQ ID NO: 5) | (SEQ ID NO: 6) | |
| | LC09AH08 | Low | 5′-TGGTGGTGAGGCTG | 5′-CCATGAGTCGGAAC | 211 |
| | | expression | TTGAGC-3′ | CTGTGC-3′ | |
| | | | (SEQ ID NO: 7) | (SEQ ID NO: 8) | |
| | LC04DC11 | Low | 5′TGGCGTTTTCTTCAT | 5′-CAGCTGCCCTTATC | 201 |
| | | expression | CCTCCA-3′ | CTGAACTGA-3′ | |
| | | | (SEQ ID NO: 9) | (SEQ ID NO: 10) | |
| | LA12AA05 | High | 5′-CGGGGTGTTGATGC | 5′-GAGGGGCTTCCATT | 210 |
| | | expression | TGAAAC-3′ | CATTATCAGA-3′ | |
| | | | (SEQ ID NO: 11) | (SEQ ID NO: 12) | |
| | LA14AD08 | High | 5′-AACCCTCGCCGGAG | 5′-TTTAATGGGATCCC | 203 |
| | | expression | CATCAA-3′ | CAACTTCTTG-3′ | |
| | | | (SEQ ID NO: 13) | (SEQ ID NO: 14) | |
| | FB14DB02 | Low | 5′-GCAATAGCTGGTCG | 5′-ATCGATTCGCTGCG | 205 |
| | | expression | GCTAGAACA-3′ | GCCTTA-3′ | |
| | | | (SEQ ID NO: 15) | (SEQ ID NO: 16) | |
| Fruit- | Les.331.1.S1_at | Fruit- | 5′-ATGTCTTTGGGTGG | 5′-CATCTCCTCGCAAA | 559 |
| specific | | specific | AATTGTGGATGCC-3′ | GCTACCAGTTC-3′ | |
| expression | | expression | (SEQ ID NO: 17) | (SEQ ID NO: 18) | |
| gene | Les.3122.2.A1_a_at | Fruit- | 5′-ATGTATGCTACGAC | 5′-CAACCCGCTGGATT | 573 |
| | | specific | CATTACTGGTAGCC-3′ | AATGAGACCAC-3′ | |
| | | expression | (SEQ ID NO: 19) | (SEQ ID NO: 20) | |
| | LesAffx.6852.1.S1_at | High in | 5′-ATGCCGCCGTTGTT | 5′-GAAAGACCAACTGA | 569 |
| | | fruit | TATCACCCATTC-3′ | GCCTCTTTCAGAAG-3′ | |
| | | but low | (SEQ ID NO: 21) | (SEQ ID NO: 22) | |
| | | in flower | | | |
| | TC115787 | No fruit- | 5′-CCACTTGTGGAATT | 5′-GATCACTTGGAGGA | 538 |
| | | specific | GGATGGGATGTTG-3′ | GCTGTATAGCC-3′ | |
| | | expression | (SEQ ID NO: 23) | (SEQ ID NO: 24) | |
| | TC116003 | No fruit- | 5′-ATGCCGCCGTTGTT | 5′-GAAAGACCAACTGA | 503 |
| | | specific | TATCACCCATTC-3′ | GCCTCTTTCAGAAG-3′ | |
| | | expression | (SEQ ID NO: 25) | (SEQ ID NO: 26) | |
| Control | E8 | Red-ripe | 5′-AACTCCATGCGGGG | 5′-GGCCTCTGTTATGG | 548 |
| | | fruit- | AGTCAT -3′ | AGGTAACT -3′ | |
| | | specific | (SEQ ID NO: 27) | (SEQ ID NO: 28) | |
| | | expression | | | |
| | Actin | Universal | 5′-AACTGGGATGATAT | 5′- TCTCAACATAATC | 141 |
| | | expression | GGAGAAGA -3′ | TGGGTCAT -3′ | |
| | | | (SEQ ID NO: 29) | (SEQ ID NO: 30) | |

Expression (mRNA) of the candidate high expression genes in mature-green fruits was first detected after the 25 cycles of the reaction, and clearly detected after the 27 cycles and the 30 cycles of the reaction (FIG. 1). The expression intensities differed among genes (FIG. 1, Table 1). On the basis of the results after the 27 cycles and the 30 cycles of the reaction, three genes LA22CD07, LA12AA05, and LA14AD08 which had exhibited particularly high expression levels were selected from among the 7 candidate high expression genes in mature-green fruits.

In contrast, as shown in FIG. 2, expression of the candidate fruit-specific expression genes was clearly detected after the 25t cycles of the reaction. Among the five candidate fruit-specific expression genes, three genes Les.3122.2.A1_a_at, LesAffx.6852.1.S1_at, and Les.331.1.S1_at which had exhibited fruit-specific expression were selected. While the E8 gene, which was known to be expressed specifically in red-ripe fruits, was expressed only in red-ripe fruits, all the three above genes were expressed in mature-green fruits in addition to red-ripe fruits.

Differences were observed in the expression patterns of these three genes. Les.3122.2.A1_a_at was expressed at high levels both in mature-green fruits and red-ripe fruits, whereas the expression level of LesAffx.6852.1.S1_a_at was higher in mature-green fruits and lower in red-ripe fruits. Les.331.1.S1_at was highly expressed in both mature-green fruits and red-ripe fruits, and it was weakly expressed in flowers.

In the Examples below, the selected three high expression genes in mature-green fruits LA22CD07, LA12AA05, and LA14AD08 and the three fruit-specific genes Les.331.1.S1_at, Les.3122.2.A1_a_at, and LesAffx.6852.1.S1_at were used as candidate genes for promoter isolation.

### Example 3

### BLAST Analysis

In order to examine the functions of the candidate genes, sequence analysis was carried out using the BLASTN programs at the National Center for Biotechnology Information (NCBI, USA).

As a result of the BLAST analysis, the gene LA14AD08 was shown to correspond to GenBank Accession Number L38581, a tomato-derived, functionally unannotated cDNA sequence) and to be a member of the Cip protease gene family. The gene LA22CD07 was shown to correspond to GenBank Accession Number AK322312, a tomato-derived, functionally unannotated cDNA sequence. LA22CD07 was also shown to be homologous to the cDNA sequence of castor bean putative erythroblast macrophage protein (emp) (GenBank Accession Number XM 002525023) (e-value=5E-39), which indicates that the gene LA22CD07 can be a homolog of the gene. In addition, the gene LA12AA05 was shown to correspond to GenBank Accession Number AK322226, a tomato-derived, functionally unannotated cDNA sequence. LA12AA05 was also shown to be homologous to the cDNA sequence of castor bean putative sum (GenBank Accession Number XM_{—}002534741) (e-value=2E-69), which indicates that the gene LA12AA05 can be a homolog of the gene.

The gene Les.331.1.S1_at (corresponding to the tomato-derived, functionally unannotated cDNA sequence of GenBank Accession Number AK326139) was found to be a tomato LOX gene that had previously been reported (GenBank Accession Number U13681). The tomato LOX gene has been reported to be expressed specifically in a fruit; and in Northern analysis, it is not expressed in mature-green fruits, but highly expressed in red-ripe fruits (Ferrie, B. J. et al., 1994, Plant Physiol., 106, 109-118). It has been reported that in promoter analysis via GUS staining, the highest staining intensity was observed in an orange-colored fruit (Beaudoin, N. and Rothstein, S. J., 1997, Plant Mol. Biol., 33, 835-46).

The gene Les.3122.2.A1_a_at was found to be a tomato pectin methylesterase-like gene (GenBank Accession Number S66607), which has previously been reported. However, the results of expression analysis thereof or promoter analysis have not yet been reported. The gene LesAffx.6852.1.S1_at did not hit to any functionally annotated tomato genes, but it showed 69% homology with the Gossypium hirsutum cysteine protease gene (GenBank Accession Number AY171099), which indicates that the gene LesAffx.6852.1.S1_at is a member of the cysteine protease gene family.

### Example 4

### Isolation of Promoter

Isolation of promoter region from each of the five genes that had not been reported about promoter analysis, i.e., LA22CD07, LA12AA05, LA14AD08, Les.3122.2.A1_a_at, and LesAffx.6852.1.S1_at, was performed as described below.

### (1) Extraction of Genomic DNA

Genomic DNA was extracted from tomato leaves (tomato cultivar: Micro-Tom or Moneymaker) by the CTAB method involving the use of cetyltrimethylammonium bromide (Nacalai Tesque, Inc.) (Murray and Thompson, 1980). Specifically, about 3 g of leaves was ground in a mortar in the presence of liquid nitrogen to prepare powder. The powder was transferred into a plastic tube containing 5 ml 2×CTAB solution at 70° C., and the tube was shaken slowly at 55° C. for 60 minutes. To this tube, 5 ml of a chloroform/isoamyl alcohol was added, and the tube was shaken slowly at room temperature for 30 minutes. Subsequently, centrifugation was carried out at 5,000 rpm for 15 minutes at room temperature, the upper layer (an aqueous phase) was transferred to a fresh plastic tube, and one-tenth of the volume of 10% CTAB solution was added thereto, followed by mixing. The mixture was shaken slowly at room temperature for 10 minutes and then centrifuged at 5,000 rpm for 15 minutes at room temperature. The upper layer (an aqueous phase) was transferred to a fresh tube, an equal amount of precipitation buffer was added thereto, the tube was inverted to mix, and the resulting mixture was incubated at room temperature for 30 minutes. After the tube was centrifuged at 5,000 rpm for 15 minutes at room temperature, the supernatant was removed. 5 ml of 1M NaCl-TE solution (containing RNase I) was added to the pellet, and the pellet was dissolved at 55° C. Isopropanol (5 ml) was added thereto, the tube was inverted to mix, and the mixture was incubated at room temperature for 30 minutes. After the resultant was centrifuged at 5,000 rpm for 15 minutes at room temperature, the supernatant was removed. After 5 ml of 70% ethanol was added to the pellet and the tube was inverted to mix, the mixture was centrifuged at 5,000 rpm for 15 minutes at room temperature. After the supernatant was removed, the pellet was roughly dried at room temperature and dissolved in a TE solution. The amount of genomic DNA was determined by measuring the OD 260 using the Safire microplate reader (Tecan, Switzerland).

### (2) Promoter Isolation and Nucleotide Sequencing

With the use of the genomic DNA as a template, upstream regions of the genes were amplified by the genome walking method using the Genome Walker™ Universal Kit (Clontech, USA). Specifically, first, the Micro-Tom-derived genomic DNA prepared above was digested with DraI, EcoRV, PvuII, or StuI restriction enzyme, and adaptor sequences were ligated to both ends of the cleavage fragments to prepare four types of genome libraries. Subsequently, primary PCR was carried out using the genome libraries as templates and the adaptor primer 1 (AP1) and the gene-specific primer 1 (GSP1). The PCR reaction was carried out in total volume of 50 μl. The reaction solution was composed of 20 pM of each primer, 1 μl of the genome library, 5 μl of 10×PCR buffer, 4 μl of dNTPs, 1.0 μl of polymerase, and 37.0 μl of sterile water. 10×PCR buffer, dNTPs, and polymerase used were from the Expand High Fidelity PCR system (Roche). PCR reaction was carried out under thermal cycling conditions of 7 cycles of denaturation at 94° C. for 0.25 minutes, annealing and extension at 72° C. for 3 minutes, and 32 cycles of denaturation at 94° C. for 0.25 minutes, annealing and extension at 67° C. for 3 minutes, followed by extension at 67° C. for 7 minutes. Subsequently, the primary PCR product was used as a template to perform secondary PCR with adaptor primer 2 (AP2) and gene-specific primer 2 (GSP2). Other composition of the reaction solution and the reaction conditions are the same as those employed for primary PCR. 5 μl of the resulting PCR product was electrophoresed on agarose gel to confirm the amplified product.

The PCR product after the secondary reaction was separated by agarose gel electrophoresis, stained with ethidium bromide, and the stained DNA fragment was excised from agarose gel, and purified using the Wizard® SV Gel and PCR Clean-Up System (Promega, USA). The nucleotide sequence of the purified DNA fragment was determined via direct sequencing. The determined nucleotide sequence was analyzed using the ORF Finder at NCBI (http://www.ncbi.nlm.nih.gov/gorf/gorf.html) to deduce ORF. In addition, the nucleotide sequence was compared with the sequences of homologs from other plant species using the BLAST program (http://blast.ncbi.nlm.nih.gov/Blast.cgi). Thus, the translation initiation site was identified.

Subsequently, in order to clone the promoter region, an approximately 2 kb upstream region of the translational initiation site was amplified from the genomic DNA of the tomato cultivar Moneymaker prepared above as a template by PCR using a primer set designed based on the nucleotide sequence information and the translation initiation site determined above, and high fidelity KOD Plus (TOYOBO, Japan). For example, the primers used for the amplification in the case of LesAffx.6852.1.S1_at were the primer 6852-5-1 (5′-GGGAAGCTTTCGTGGAAACTATCTTTCACG-3; SEQ ID NO: 31) which includes the restriction enzyme HindIII recognition sequence 5′-AAGCTT-3′ added on the 5′ side, and the primer 6852-3-1 (5′-GGGGTCTAGATTTTCAGTTACATTAAACAGTTATTG-3′; SEQ ID NO: 32) which includes the restriction enzyme XbaI recognition sequence 5′-TCTAGA-3′ added on the 5′ side. Further, for example, the primers used for the amplification in the case of LA22CD07 were the primer LA22CD07-5-1 (5′-ATGCAAGCTTCGTGCGTTGCACG-3; SEQ ID NO: 33) which includes the restriction enzyme HindIII recognition sequence 5′-AAGCTT-3′ added on the 5′ side, and the primer LA22CD07-3-1 (5′-ATGCGGATCCTAATGGAAGAAATCAAG-3; SEQ ID NO: 34) which includes the restriction enzyme BamHI recognition sequence 5′-GGATCC-3′ added on the 5′ side. The PCR reaction was carried out in total volume of 50 μl. The reaction solution was composed of 20 μM of each primers of the primer set, 1 μl of genomic DNA, 5 μl of 10×PCR buffer, 5 μl of dNTPs, 3.0 μl of MgCl₂, 1.0 μl of KOD-Plus polymerase, and 34.6 μl of sterile water. 10×PCR buffer and dNTPs used were reagents attached to Poly KOD-plus- (TOYOBO). PCR reaction was carried out under thermal conditions of thermal denaturation at 94° C. for minutes, followed by 30 cycles of denaturation at 94° C. for 0.25 minutes, annealing at 55° C. for 0.5 minutes, and extension at 68° C. for 3 minutes.

The PCR product after the reaction was separated by agarose gel electrophoresis, stained with ethidium bromide, and the stained DNA fragment was excised from agarose gel, followed by purification using the Wizard® SV Gel and PCR Clean-Up System (Promega, USA). The purified DNA fragment was integrated into the pCR®-Blunt II-TOPO® vector using the Zero Blunt® TOPO® PCR cloning kit (Invitrogen, USA). The nucleotide sequences of the DNA fragment cloned into the resulting plasmid vector were determined by a conventional technique.

Representative examples of the nucleotide sequences of the promoter regions in the cloned DNA fragments are shown in SEQ ID NO: 1 (promoter sequence of LA22CD07) and SEQ ID NO: 2 (promoter sequence of LesAffx.6852.1.S1_at).

### Example 5

### Gene Expression Assay

(1) Construction of Plant Expression Vector and Gene Introduction into Agrobacterium

Promoter activity was evaluated based on GUS gene expression activity. At the outset, the DNA fragment comprising the promoter region of each gene was inserted into pBI121, which is a plant expression vector comprising GUS gene, in the following manner. The promoter region integrated into the pCR®-Blunt II-TOPO® vector prepared in Example 4 was excised with restriction enzymes, and the excised DNA fragment was inserted into a site upstream of the GUS gene in the pBI121 expression vector from which the 35S promoter had been removed. The constructed vector was introduced into Agrobacterium tumefaciens GV3101 strain via electroporation, and the transformed Agrobacterium was selected on a LB agar medium supplemented with antibiotic kanamycin at 50 mg/l.

### (2) Transient Expression Analysis

Transient expression analysis using tomato mature-green fruits was carried out in accordance with the method of Orzaez et al. (2006). Specifically, the transformed Agrobacterium produced above was cultured at 28° C. overnight in 5 ml of a liquid YEB medium supplemented with antibiotic kanamycin at 50 mg/l, and then further cultured overnight in 50 ml of an induction medium (0.5% meat extract (beef extract), 0.1% yeast extract, 0.5% peptone, 0.5% sucrose, 2 mM MgSO₄, 20 μM acetosyringone, 10 mM MES, pH 5.6) supplemented with antibiotic kanamycin at 50 mg/l. The culture solution was centrifuged at 3,000 rpm for 10 minutes at room temperature, followed by harvesting. The harvested cell pellets were suspended in an infection medium (10 mM MgCl₂, 10 mM MES, 200 μM acetosyringone, pH 5.6) at a concentration of OD 600=1.0, and the medium was allowed to slowly permeate at room temperature for 2 hours. The bacteria solution (500 μl) was transferred to a 1-ml syringe, and a needle of the syringe containing the solution was inserted into mature-green fruits removed from Micro-Tom to inject the bacteria solution into the fruits. The fruits were placed in a 9-cm plastic petri dish containing a filter paper immersed in 2 ml of distilled water, and then incubated at 25° C. for 4 days with 16 hours day length.

Subsequently, the total protein was extracted from the fruit and quantitative GUS activity assay was carried out using 4-MUG as a substrate. The GUS activity assay technique of Jefferson et al. (1987) using 4-MUG as a substrate was employed for the quantitative assay with slight modifications. Specifically, the mature-green fruits infected with the transformed Agrobacterium and then incubated for 4 days as described above were pulverized with liquid nitrogen, and the total protein was extracted therefrom using the protein extraction buffer (100 mM phosphate buffer (pH 8.0), 10 mM EDTA, 0.1% Triton X-100, 0.1% sarcosyl, 10 mM mercaptoethanol). With the use of the Quick Start Protein Assay Kit (Bio-Rad), the protein amount in the extracted sample was measured by the Bradford method (Bradford, 1976). Serial dilutions of the extracted total protein and of the bovine serum albumin as a standard protein were prepared. The dilutions (5 μl each) were added to 250 μl of dye solution, and the resultants were incubated at room temperature for 10 minutes. The absorbance at 595 nm was assayed using Safire (Tecan, Switzerland). On the basis of the calibration curve prepared with bovine serum albumin and the absorbance of the extract sample, the protein amount in the extracted sample was calculated.

Approximately 100 μg of the extracted proteins were used for GUS activity assay. Approximately 100 μg of the extracted proteins, 50 μl of 20 mM 4-methyl-umbelliferyl-β-D-glucuronide (4-MUG) and the same protein extraction buffer used above were added to a 1.5-ml plastic tube on ice to a total volume of 1 ml, followed by mixing. The reaction solution was incubated at 37° C. for 0, 2, and 4 hours, a 200-μl was sampled at the completion of each reaction period, and the reaction was terminated with the addition of 0.8 ml of a reaction terminator solution (0.2 M Na₂CO₃). In order to measure the amount of the reaction product 4-methylumbelliferone (4-MU), the fluorescence at 455 nM with excitation at 365 nM was assayed for each time samples using Safire (Tecan, Switzerland). The measured values were compared with the calibration curve prepared for 4-MU to determine the amount of 4-MU in the samples. GUS activity was represented by the amount of 4-MU (pmole/min/mg protein), and then GUS activity was evaluated.

As a result, the promoters of the three genes LA22CD07, Les.3122.2.A1_a_at, and LesAffx.6852.1.S1_at were found to have activity to induce transient gene expression in fruits.

### Example 6

### Production of Transgenic Tomato and Expression Analysis Thereof

Transformants of Micro-Tom were produced by the method of Sun et al. (Plant Physiol., 114: 1547-1556, 2006), using the transformed Agrobacterium produced in Example 5(1) which were obtained by introducing the vectors comprising the promoter regions of the genes confirmed to exert activity in fruits (LA22CD07, Les.3122.2.A1_a_at, and LesAffx.6852.1.S1_at) into the A. tumefaciens GV3101 strain. As a control, using a transformed Agrobacterium into which a pBI121 expression vector comprising the GUS gene under the control of the 35S promoter is introduced, a transformant of Micro-Tom were produced in a similar way.

The transformed Agrobacterium were cultured with shaking overnight in LB medium supplemented with antibiotic kanamycin at 50 mg/l. The culture solution was centrifuged at 3,000 rpm for 10 minutes at room temperature, and the supernatant was removed. The harvested cell pellets were washed and then suspended in liquid MS medium supplemented with 200 μM acetosyringone and 10 μM mercaptoethanol at a concentration of OD 600=1.0. In the Agrobacterium solution, Slices of aseptic Micro-Tom cotyledon 7 days after aseptic inoculation were immersed. The tomato cotyledon slices thus infected with Agrobacterium were co-cultured in MS medium supplemented with 1.5 mg/l zeatin for 3 days. Thereafter, the cultured slices were transferred to a selection MS medium supplemented with 1 mg/l zeatin and 100 mg/l kanamycin, culture was continued while exchanging media every two weeks, and the grown shoots were transferred to a rooting MS medium supplemented with 50 mg/l kanamycin to develop roots.

Genomic DNA was extracted from the leaves of regenerated plants that had developed roots in the rooting MS medium in the manner described above. GUS gene was amplified by PCR using the extracted genomic DNA as a template. The primers used are GUS-F primer (5′-GATCAGTTCGCCCATGCAGATATTCG-3; SEQ ID NO: 35) and GUS-R primer (5′-CTTGCAAAGTCCCGCTAGTGCC-3′; SEQ ID NO: 36). The PCR reaction was carried out in total volume of 20 μl. The reaction solution was composed of 20 pM of each primer, 1 μl of genomic DNA, 2 μl of 10×PCR buffer, 1.6 μl of dNTPs, 0.4 μl of polymerase, and 15.8 μl of sterile water. 10×PCR buffer, dNTPs, and polymerase used were of the TAKARA Taq Hot Start Version. PCR reaction was carried out under thermal cycling conditions of thermal denaturation at 95° C. for 5 minutes, followed by 30 cycles of denaturation at 94° C. for 0.5 minutes, annealing at 55° C. for 0.5 minutes, and extension at 72° C. for 2 minute. The resulting PCR product (5 μl) was electrophoresed on agarose gel, the amplified product was examined, and regenerated plants in which GUS gene amplification was observed were selected as transformants.

The thus produced primary transformant plants (transgenic plants) were assayed for the expression induction activity in different tissues of the promoters via GUS staining. The technique of Jefferson et al. (EMBO J., 6: 3901-3907, 1987) using X-GLUC (5-brom-4-chloro-β-indolyl-β-D-gluculonic acid) as a substrate was employed for the GUS staining-based expression analysis with slight modifications. Specifically, 50 mM phosphate buffer (pH 7.0) in the reaction solution was replaced with 100 mM phosphate buffer (pH 8.0), and the resulting reaction solution (1 mM X-Gluc, 0.5 mM potassium ferricyanide, 0.5 mM potassium ferrocyanide, 100 mM phosphate buffer, pH 8.0) was used in order to reduce background staining. Since background signals were detected for red-ripe fruits even using the above-mentioned reaction solution, staining with the above-mentioned reaction solution supplemented with methanol at final concentration of 20% according to the method of Kosugi et al. (Plant Sci., 70:133-140, 1990) was also carried out for further reduction of background staining for red-ripe fruits. Staining was carried out by immersing the tissue samples obtained from the transformed tomatoes in the reaction solution, allowing the dye to permeate the tissues under reduced pressure for 15 minutes, and incubating at 37° C. overnight (16 hours) or for 6 hours (exclusively for the reaction solution supplemented with 20% methanol). After the incubation, the samples were washed with 70% ethanol to terminate the reaction.

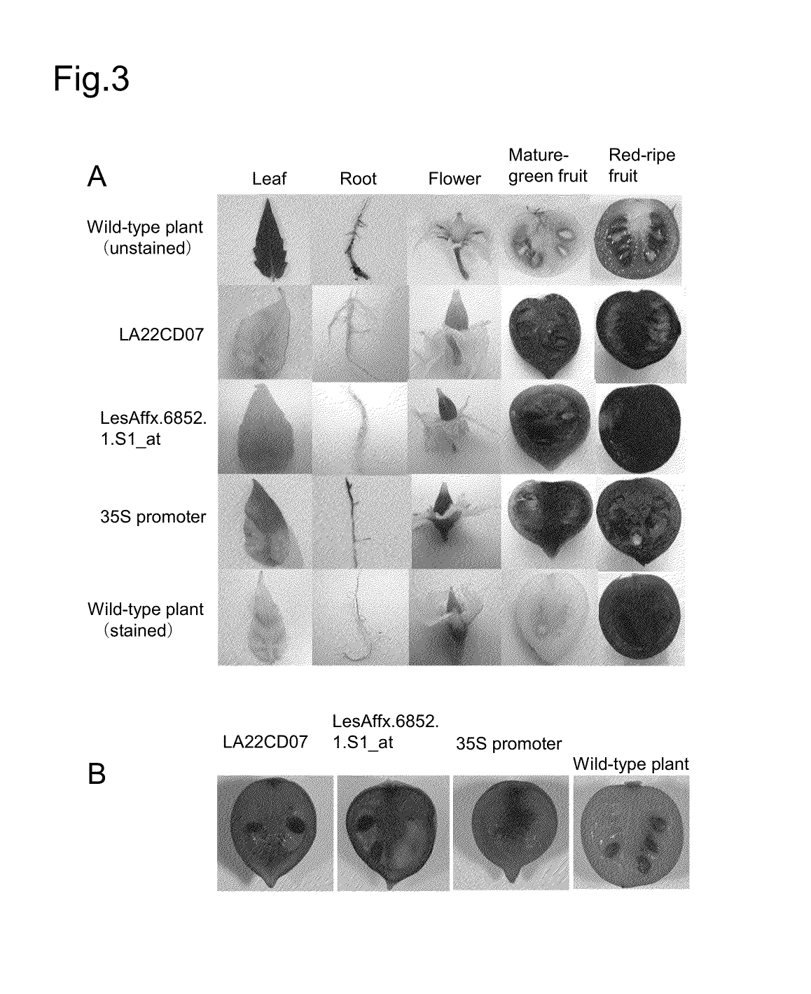

An example of the result is shown in FIG. 3. FIG. 3A shows the result of staining the tissue of the transformant immersed overnight in the reaction solution with 100 mM phosphate buffer (pH 8.0) instead of 50 mM phosphate buffer (pH 7.0). FIG. 3B shows the result of staining the red-ripe fruits of the transformant immersed in the reaction solution supplemented with 20% methanol for 6 hours.

As shown in FIG. 3, among the three tested promoters, the promoters of LA22CD07 and LesAffx.6852.1.S1_at had activity in the transformants, and both these promoters showed intense GUS staining particularly in fruits. The promoter of LA22CD07 showed intense staining in mature-green fruits and red-ripe fruits and weak staining in flowers, whereas no staining was observed in other tissues (leaves and roots). While the promoter of LesAffx.6852.1.S1_at also showed intense staining in mature-green fruits and red-ripe fruits, the staining intensity thereof in flowers was weaker than that of the promoter of LA22CD07, and no staining was observed in other tissues (leaves and roots). The promoter of Les.3122.2.A1 did not show any GUS staining in any of the tested tissue samples.

In contrast, the 35S promoter showed GUS staining in all the tested tissue samples. In wild-type plants (i.e., non-transformants), no GUS staining was observed in mature-green fruits, flowers, leaves, or stems. On the other hand, as shown in FIG. 3A, staining was observed in red-ripe fruits of wild-type plants, and the background signal level (non-specific staining intensity) was found to be high in red-ripe fruits. Thus, to reduce the background signals, GUS staining was carried out with the use of the reaction solution supplemented with 20% methanol (FIG. 3B). As shown in FIG. 3B, substantially no staining was observed in red-ripe fruits of wild-type plants; however, the promoters of LesAffx.6852.1.S1_at and LA22CD07, and the 35S promoter showed staining in red-ripe fruits.

Accordingly, the promoters of the two genes LesAffx.6852.1.S1_at and LA22CD07 were shown to strongly induce gene expression, and therefore to have strong promoter activity, in mature-green fruits in addition to red-ripe fruits. Also, the promoters of LesAffx.6852.1.S1_at and LA22CD07 were found to have weak activity in flowers.

The above results have revealed that, unlike the conventional plant promoter (E8) that functions only at the late developmental stage of fruits, the promoters of LA22CD07 and LesAffx.6852.1.S1_at function even in mature-green fruits at the early developmental stage of fruits, in addition to red-ripe fruits. Since gene expression was also observed in flowers, further, such promoters were found to begin to function at the earliest developmental stage of fruits from flowers to mature-green fruits.

Further, BLASTN search was performed with cDNA sequences corresponding to LesAffx.6852.1.S1_at and LA22CD07. As a result, homolog genes of a variety of plants other than tomatoes (e.g., cotton (Gossypium hirsutum)) were hit. This indicates that the promoters of LesAffx.6852.1.S1_at and LA22CD07 can also function in the same manner in fruits of other plant species.

### INDUSTRIAL APPLICABILITY

Unlike conventional E8 promoters and the like, the promoter according to the present invention exerts strong gene expression-inducing activity in mature-green fruits in addition to red-ripe fruits of, for example, tomato. Accordingly, the promoters according to the present invention can overcome the drawback of conventional fruit-specific promoters that they function only at the late developmental stage, and thus they can be preferably used to induce expression of foreign genes, regardless of the developmental stages of fruits. For example, the induction of foreign gene expression starting from the mature-green stage, which is the early developmental stage, can shorten the duration required for recombinant protein production in plants, or enables recombinant protein production for an extended period of time. The promoter of the present invention is also useful for production of, for example, proteins that are more easily purified from mature-green tomato fruits than from red-ripe tomato fruits. The promoter of the present invention is also capable of expressing proteins that would adversely affect plants when expressed in leaves or stems, at high levels in fruits while avoiding such adverse effects.

### Sequence Listing Free Text

SEQ ID NO: 1: LA22CD07 promoter
SEQ ID NO: 2: LesAffx.6852.1.S1_at promoter
SEQ ID NOs: 3 to 36: primers

1. A fruit-specific promoter DNA, which consists of a nucleotide sequence having 80% or more identity with the nucleotide sequence as shown in SEQ ID NO: 1 or 2 and has promoter activity in mature-green fruits.

2. The fruit-specific promoter DNA according to claim 1, which consists of a nucleotide sequence having 90% or more identity with the nucleotide sequence as shown in SEQ ID NO: 1 or 2.

3. An expression vector comprising the fruit-specific promoter DNA according to claim 1.

4. The expression vector according to claim 3, which further comprises a gene ligated downstream of said fruit-specific promoter DNA.

5. A DNA construct comprising the fruit-specific promoter DNA according to claim 1 and a gene ligated downstream thereof.

6. A transformed cell comprising the expression vector according to claim 3.

7. A transformed plant into which the expression vector according to claim 4 is introduced.

8. A method for producing a transformed plant comprising introducing the expression vector according to claim 4 into a plant cell, growing the transformed plant to develop a fruit, and confirming the expression of said gene in the fruit.

9. A method for producing a recombinant gene product comprising growing the transformed plant according to claim 7 to develop a fruit and obtaining the expressed gene product from the fruit.

10. An expression vector comprising the fruit-specific promoter DNA according to claim 2.

11. A DNA construct comprising the fruit-specific promoter DNA according to claim 2 and a gene ligated downstream thereof.

12. A transformed cell comprising the expression vector according to claim 4.

13. A transformed cell comprising the DNA construct according to claim 5.

14. A transformed plant into which the DNA construct according to claim 5 is introduced.

15. A method for producing a transformed plant comprising introducing the DNA construct according to claim 5 into a plant cell, growing the transformed plant to develop a fruit, and confirming the expression of said gene in the fruit.
